SEQUENCE LISTING (5 sequences)

SEQ ID NO: 1 - protein, 231 aa, Thalassiosira pseudonana
MKTSAIALLAVLATTAATEPRRLRTLEGHGGDHSISMSMHSSKAEKQAIEAAVEEDVAGPAKAAKLFKPKASKAGSMPDEAGAKSAKMSMDTKSGKSEDAAAVDAKASKESHMSISGDMSMAKSHKAEAEDVTEMSMAKAGKDEASTEDMCMPFAKSDKEMSVKSKQGKTEMSVADAKASKESSMPSSKAAKIFKGKSGKSGSLSMLKSEKASSAHSLSMPKAEKVHSMSA

SEQ ID NO: 2 - protein, 501 aa, Thalassiosira pseudonana
MKVTTSIITLLFASCGAADVQRVLEDVTEPAVTTPAATPAPITPEPATPAPTICEGRNFYYDEETRKCSNEATGGIYGTLIDCCVAISGSVSCPYVDICNTLQPSPSPETNEPSAKPITAAPISSAPVSAAPVTSAPVAAPVETTSMTGPTTIVASIVSTNAPSLTNAPSSSLEAVVTRIPVETTNTASPTTTAASIVSTNAPSSSPEAVVTPRPTFRPSPEGTESNTSPASIASDVMFGPPKTSTPTSTPTSSSHPSSSEPTLSPSVSKEPTGYPTSSPSHSPTKSPSKSPSSSPTTSPSASPTETPTETPTESPTESPTESPTLSPTESPTLSPTESPSLSPTLSTTWSPTGYPTLAPSPSPSISSAPSVSSAPSSPPSISSAPSVSSAPSKNFGFLPGLTEMPTISPTEDHYFFGKSHKSHKSHKSKATKTLKVSKSGKSAKSSKSSGRRPLFGVSQLSEGIAVGYAKSSGRSSQQAVGSWMPVAAACILGALSFFLN

SEQ ID NO: 3 - protein, 485 aa, Thalassiosira pseudonana
MKVTTSIITLLFASCGAADVQRVLEDVTEPAVTTPAATPAPITPEPATPAPTICEGRNFYRDDDTGKCSNEATGGIYGTLIECCVAISGSDSCPYVDICNTLQPSPSPETNEPSAKPITAAPISSAPVSAAPVTSAPVAAPVETTSMTGPTTIVASIVSTNAPSSTNAPSSSLEAVVTRIPVETTNTASPTTTAASIVSTNAPSSSPEAVVTPRPTFRPSPKGTESNTFPASIASDVMFDPARSEPTFTPTSSSQPSSSEPTLSPSVSKEPTRYPTSSPSHSPTKSPSKSPSSSPTTSPSASPTETPTETPTESPTELPTLSPTEFPSLSPTLSPTWSPTGYPTLAPSPSPSISSAPSVSSAPSSSPSISSAPSVSSAPSKNFGFLPGRNEMPTISPTEDHYFFGKSHKSHKSKATKTLKVSKSGKSSKSSKSSGRRPLFGVSQLSEGIAAGYAKSSGRSSQQAVGSWMPVAAACILGALSFFLN

SEQ ID NO: 4 - protein, 20 aa, Artificial Sequence
  synthetic silaffin protein
VKVKVKVKVPPTKVEVKVKV

SEQ ID NO: 5 - protein, 20 aa, Artificial Sequence
  alternate synthetic silaffin protein
VKVSVKVSVPPTKVSVKVSV

(19) United States
(12) Patent Application Publication
(10) Pub. No.: US 2013/0029397 A1
(43) Pub. Date: Jan. 31, 2013

(54) MATERIALS AND METHODS FOR PRODUCING CELL-SURFACE DIRECTED AND ASSOCIATED NON-NATURALLY OCCURRING BIOINORGANIC MEMBRANCES AND USES THEREOF

(71) Applicants: David Benjamin Jaroch, Tonawanda, NY (US); Jenna Leigh Rickus, West Lafayette, IN (US)

(72) Inventors: David Benjamin Jaroch, Tonawanda, NY (US); Jenna Leigh Rickus, West Lafayette, IN (US)

(73) Assignee: Purdue Research Foundation, West Lafayette, IN (US)

(21) Appl. No.: 13/546,834

(22) Filed: Jul. 11, 2012

Related U.S. Application Data
(63) Continuation of application No. PCT/US2011/021032, filed on Jan. 12, 2011.
(60) Provisional application No. 61/294,209, filed on Jan. 12, 2010.

(51) Int. Cl. C12N 11/14

(52) U.S. Cl. 435/176

(57) ABSTRACT

Materials and methods are provided for producing cell-surface directed, non-naturally occurring, bioinorganic membranes for association with the cell surfaces of living cells. The methods comprise exposing a cell to an acidic biomineralization buffer environment for cell-mediated deposition of the biomineral membrane onto the surface of the cell. The methods also comprise attaching a peptide, having a net positive charge under the acidic conditions, to the cell surface for serving as a template in directing the cell-mediated deposition of the biomineral membrane onto the surface of the cell.

### PRIORITY CLAIM

This application is a continuation of International Application No. PCT/US2011/021032, filed Jan. 12, 2011, which claims the benefit of U.S. Provisional Patent Application No. 61/294,209, filed Jan. 12, 2010, the disclosures of which are hereby incorporated by reference in their entirety.

### STATEMENT OF GOVERNMENTAL RIGHTS

This invention was made with government support under grant number RR025761 awarded by the National Institute of Health (NIH), and under grant number W911NF-09-1-0447 awarded by the U.S. Army Research Laboratory's Army Research Office (ARO). The U.S. government has certain rights in the invention.

### FIELD OF THE DISCLOSURE

The present disclosure relates generally to biomineralization of a membrane at the cell surface of living cells. More particularly, the present disclosure relates to producing cell-surface directed and associated non-naturally occurring bioinorganic membranes with living cells.

### BACKGROUND

During evolution, some classes of living cellular organisms developed the ability to manipulate inorganic materials. Diatoms, a large class of eukaryotic unicellular algae believed to have originated prior to the Jurassic period, are one such organism. Diatoms have cell walls comprised of silica and are capable of forming diverse inorganic and hybrid materials with unique functionality and complex nano and micro-scale architectural features. In general, organisms capable of forming bioinorganic membranes (such as diatoms) form these membranes by producing a matrix (generally a protein matrix) which serves as a template for the deposition of the bioinorganic membrane and by manipulating the chemical composition of cellular microenvironments.

To date, artificial deposition strategies for classes of organisms which are evolutionary distinct from organisms such as diatoms have yet to produce bioinorganic membranes which rival the functionality and structural features possessed by the cell walls of diatoms. Traditionally, cell immobilization methods result in cell entrapment within bulk materials, creating significant diffusion barriers hindering survival of the cell. Also, current technology (generally based on passive silica deposition) creates thin coatings with poor mechanical strength around the cells which are brittle amorphous structures that degrade and are poorly resistant to various physiological fluids over time.

Further, current artificial deposition strategies result in the formation of membranes having indiscriminate pore morphology which tends to cause slower molecular diffusion into and out of the cell. Pore morphology, however, is an important feature for the viability of cells having associated cell surface bioinorganic membranes. For cells to remain viable, the associated bioinorganic membrane must allow the free diffusion of small molecules while excluding the passage of other large molecules and cells.

Therefore, it would be desirable to have a method for producing and associating a non-naturally occurring bioinorganic membrane with a cell surface of a living cell which allows for the design and control of pore morphology.

### SUMMARY OF THE DISCLOSURE

The present disclosure provides methods for forming a bioinorganic membrane by attaching at least one polypeptide to a surface of at least one cell, wherein the cell does not form a bioinorganic membrane in nature, and wherein said at least one polypeptide associates with a non-naturally occurring bioinorganic material, wherein said non-naturally occurring bioinorganic material is rich in silica.

In certain embodiments, the cell is a eukaryotic cell, a pancreatic beta cell, or a prokaryotic cell, such as a prokaryotic cell from one species of the genus Pseudomonas.

In certain embodiments, the method further includes exposing the cells to a biomineralization solution, wherein the solution is mildly acidic and rich in silica. The biomineralization solution may be low in methanol and formed by hydrolyzing tetramethyl orthosilicate in an acid.

The present disclosure also provides methods for forming a bioinorganic membrane by attaching a polypeptide to a surface of a bio-film, wherein the bio-film does not form a bioinorganic membrane in nature, and wherein said at least one polypeptide associates with a non-naturally occurring bioinorganic material, wherein said non-naturally occurring bioinorganic material is rich in silica.

In certain embodiments, the bio-film is a surface of a pancreatic islet.

In certain embodiments of these methods, said polypeptide is attached directly to the surface of the cell or the bio-film. For example, the polypeptide may be bound, link, or associated with at least one group, wherein said at least one group is part of the cell or the bio-film.

In other embodiments of these methods, said polypeptide is attached indirectly to the surface of the cell or the bio-film. For example, wherein the surface of the cell or the bio-film is attached to a ligand, wherein the ligand includes a reactive group, wherein the reactive group of the ligand binds to an intermediate group, and wherein the intermediate group includes a first portion and a second portion, the first portion of the intermediate group may be attached to the reactive group of the ligand and the second portion of the intermediate group may be attached to said polypeptide.

The present disclosure further provides a bio-structure including at least one cell, at least one polypeptide, and a non-naturally occurring bioinorganic membrane. A first portion of said polypeptide may be attached either directly or indirectly to a surface of the cell. A second portion of said polypeptide is associated with a form of silica, wherein said polypeptide and the form of silica form part of the non-naturally occurring bioinorganic membrane.

The present disclosure still further provides a bio-structure including at least one bio-film, at least one polypeptide, and a non-naturally occurring bioinorganic membrane. A first portion of said polypeptide may be attached attached either directly or indirectly to a surface of the bio-film. A second portion of said polypeptide is associated with a form of silica, wherein said polypeptide and the form of silica form part of the non-naturally occurring bioinorganic membrane.

In certain embodiments, said polypeptide is selected from the group consisting of: a silicatein protein, a naturally occurring polyamine rich peptide, a non-naturally occurring polyamine rich peptide, a derivate of a silicatein, a derivative of a silaffin, a thiolated peptide, and a polypeptide that includes at least one free hydroxyl group. The polypeptide that includes at least one free hydroxyl group may include at least one amino acid selected from the group consisting of: serine, threonine, and hydroxyproline.

In certain embodiments, said polypeptide is a silaffin. The silaffin may be derived from at least one species selected from the genera consisting of: Thalassiosira and Coscinodiscus. The silaffin may be derived from at least one species selected from the group consisting of: Thalassiosira pseudonana, Coscinodiscus wailesii, and Coscinodiscus concinnus.

In certain embodiments, said polypeptide is selected from the group consisting of: SEQ ID NO. 1, SEQ ID NO. 2, SEQ ID NO.3, SEQ ID NO4, and SEQ ID NO. 5.

These and other features, aspects, and advantages of the present invention will become better understood with reference to the following drawings, descriptions and claims.

SEQUENCE LISTING

| SEQ ID | | |
|---|---|---|
| NO. | LISTING | DESCRIPTION |
| 1 | Met Lys Thr Ser Ala Ile Ala Leu Leu Ala Val Leu Ala Thr Thr Ala | Silaffin protein derived |
| | Ala Thr Glu Pro Arg Arg Leu Arg Thr Leu Glu Gly His Gly Gly | from Thalassiosira |
| | Asp | pseudonana |
| | His Ser Ile Ser Met Ser Met His Ser Ser Lys Ala Glu Lys Gln Ala | |
| | Ile Glu Ala Ala Val Glu Glu Asp Val Ala Gly Pro Ala Lys Ala Ala | |
| | Lys Leu Phe Lys Pro Lys Ala Ser Lys Ala Gly Ser Met Pro Asp Glu | |
| | Ala Gly Ala Lys Ser Ala Lys Met Ser Met Asp Thr Lys Ser Gly Lys | |
| | Ser Glu Asp Ala Ala Ala Val Asp Ala Lys Ala Ser Lys Glu Ser His | |
| | Met Ser Ile Ser Gly Asp Met Ser Met Ala Lys Ser His Lys Ala Glu | |
| | Ala Glu Asp Val Thr Glu Met Ser Met Ala Lys Ala Gly Lys Asp | |
| | Glu | |
| | Ala Ser Thr Glu Asp Met Cys Met Pro Phe Ala Lys Ser Asp Lys | |
| | Glu | |
| | Met Ser Val Lys Ser Lys Gln Gly Lys Thr Glu Met Ser Val Ala Asp | |
| | Ala Lys Ala Ser Lys Glu Ser Ser Met Pro Ser Ser Lys Ala Ala Lys | |
| | Ile Phe Lys Gly Lys Ser Gly Lys Ser Gly Ser Leu Ser Met Leu Lys | |
| | Ser Glu Lys Ala Ser Ser Ala His Ser Leu Ser Met Pro Lys Ala Glu | |
| | Lys Val His Ser Met Ser Ala | |
| 2 | Met Lys Val Thr Thr Ser Ile Ile Thr Leu Leu Phe Ala Ser Cys Gly | Silaffin protein derived |
| | Ala Ala Asp Val Gln Arg Val Leu Glu Asp Val Thr Glu Pro Ala Val | from Thalassiosira |
| | Thr Thr Pro Ala Ala Thr Pro Ala Pro Ile Thr Pro Glu Pro Ala Thr | pseudonana |
| | Pro Ala Pro Thr Ile Cys Glu Gly Arg Asn Phe Tyr Tyr Asp Glu Glu | |
| | Thr Arg Lys Cys Ser Asn Glu Ala Thr Gly Gly Ile Tyr Gly Thr Leu | |
| | Ile Asp Cys Cys Val Ala Ile Ser Gly Ser Val Ser Cys Pro Tyr Val | |
| | Asp Ile Cys Asn Thr Leu Gln Pro Ser Pro Ser Pro Glu Thr Asn Glu | |
| | Pro Ser Ala Lys Pro Ile Thr Ala Ala Pro Ile Ser Ser Ala Pro Val | |
| | Ser Ala Ala Pro Val Thr Ser Ala Pro Val Ala Ala Pro Val Glu Thr | |
| | Thr Ser Met Thr Gly Pro Thr Thr Ile Val Ala Ser Ile Val Ser Thr | |
| | Asn Ala Pro Ser Leu Thr Asn Ala Pro Ser Ser Ser Leu Glu Ala Val | |
| | Val Thr Arg Ile Pro Val Glu Thr Thr Asn Thr Ala Ser Pro Thr Thr | |
| | Thr Ala Ala Ser Ile Val Ser Thr Asn Ala Pro Ser Ser Ser Pro Glu | |
| | Ala Val Val Thr Pro Arg Pro Thr Phe Arg Pro Ser Pro Glu Gly Thr | |
| | Glu Ser Asn Thr Ser Pro Ala Ser Ile Ala Ser Asp Val Met Phe Gly | |
| | Pro Pro Lys Thr Ser Thr Pro Thr Ser Thr Pro Thr Ser Ser Ser His | |
| | Pro Ser Ser Ser Glu Pro Thr Leu Ser Pro Ser Val Ser Lys Glu Pro | |
| | Thr Gly Tyr Pro Thr Ser Ser Pro Ser His Ser Pro Thr Lys Ser Pro | |
| | Ser Lys Ser Pro Ser Ser Ser Pro Thr Thr Ser Pro Ser Ala Ser Pro | |
| | Thr Glu Thr Pro Thr Glu Thr Pro Thr Glu Ser Pro Thr Glu Ser Pro | |
| | Thr Glu Ser Pro Thr Leu Ser Pro Thr Glu Ser Pro Thr Leu Ser Pro | |
| | Thr Glu Ser Pro Ser Leu Ser Pro Thr Leu Ser Thr Thr Trp Ser Pro | |
| | Thr Gly Tyr Pro Thr Leu Ala Pro Ser Pro Ser Pro Ser Ile Ser Ser | |
| | Ala Pro Ser Val Ser Ser Ala Pro Ser Ser Pro Pro Ser Ile Ser Ser | |
| | Ala Pro Ser Val Ser Ser Ala Pro Ser Lys Asn Phe Gly Phe Leu Pro | |
| | Gly Leu Thr Glu Met Pro Thr Ile Ser Pro Thr Glu Asp His Tyr Phe | |
| | Phe Gly Lys Ser His Lys Ser His Lys Ser His Lys Ser Lys Ala Thr | |
| | Lys Thr Leu Lys Val Ser Lys Ser Gly Lys Ser Ala Lys Ser Ser Lys | |
| | Ser Ser Gly Arg Arg Pro Leu Phe Gly Val Ser Gln Leu Ser Glu Gly | |
| | Ile Ala Val Gly Tyr Ala Lys Ser Ser Gly Arg Ser Ser Gln Gln Ala | |
| | Val Gly Ser Trp Met Pro Val Ala Ala Ala Cys Ile Leu Gly Ala Leu | |
| | Ser Phe Phe Leu Asn | |
| 3 | Met Lys Val Thr Thr Ser Ile Ile Thr Leu Leu Phe Ala Ser Cys Gly | Silaffin protein derived |
| | Ala Ala Asp Val Gln Arg Val Leu Glu Asp Val Thr Glu Pro Ala Val | from Thalassiosira |
| | Thr Thr Pro Ala Ala Thr Pro Ala Pro Ile Thr Pro Glu Pro Ala Thr | pseudonana |
| | Pro Ala Pro Thr Ile Cys Glu Gly Arg Asn Phe Tyr Arg Asp Asp Asp | |
| | Thr Gly Lys Cys Ser Asn Glu Ala Thr Gly Gly Ile Tyr Gly Thr Leu | |
| | Ile Glu Cys Cys Val Ala Ile Ser Gly Ser Asp Ser Cys Pro Tyr Val | |
| | Asp Ile Cys Asn Thr Leu Gln Pro Ser Pro Ser Pro Glu Thr Asn Glu | |
| | Pro Ser Ala Lys Pro Ile Thr Ala Ala Pro Ile Ser Ser Ala Pro Val | |
| | Ser Ala Ala Pro Val Thr Ser Ala Pro Val Ala Ala Pro Val Glu Thr | |
| | Thr Ser Met Thr Gly Pro Thr Thr Ile Val Ala Ser Ile Val Ser Thr | |
| | Asn Ala Pro Ser Ser Thr Asn Ala Pro Ser Ser Ser Leu Glu Ala Val | |
| | Val Thr Arg Ile Pro Val Glu Thr Thr Asn Thr Ala Ser Pro Thr Thr | |
| | Thr Ala Ala Ser Ile Val Ser Thr Asn Ala Pro Ser Ser Ser Pro Glu | |
| | Ala Val Val Thr Pro Arg Pro Thr Phe Arg Pro Ser Pro Lys Gly Thr | |
| | Glu Ser Asn Thr Phe Pro Ala Ser Ile Ala Ser Asp Val Met Phe Asp | |
| | Pro Ala Arg Ser Glu Pro Thr Phe Thr Pro Thr Ser Ser Ser Gln Pro | |
| | Ser Ser Ser Glu Pro Thr Leu Ser Pro Ser Val Ser Lys Glu Pro Thr | |
| | Arg Tyr Pro Thr Ser Ser Pro Ser His Ser Pro Thr Lys Ser Pro Ser | |
| | Lys Ser Pro Ser Ser Ser Pro Thr Thr Ser Pro Ser Ala Ser Pro Thr | |
| | Glu Thr Pro Thr Glu Thr Pro Thr Glu Ser Pro Thr Glu Leu Pro Thr | |
| | Leu Ser Pro Thr Glu Phe Pro Ser Leu Ser Pro Thr Leu Ser Pro Thr | |
| | Trp Ser Pro Thr Gly Tyr Pro Thr Leu Ala Pro Ser Pro Ser Pro Ser | |
| | Ile Ser Ser Ala Pro Ser Val Ser Ser Ala Pro Ser Ser Ser Pro Ser | |
| | Ile Ser Ser Ala Pro Ser Val Ser Ser Ala Pro Ser Lys Asn Phe Gly | |
| | Phe Leu Pro Gly Arg Asn Glu Met Pro Thr Ile Ser Pro Thr Glu Asp | |
| | His Tyr Phe Phe Gly Lys Ser His Lys Ser His Lys Ser Lys Ala Thr | |
| | Lys Thr Leu Lys Val Ser Lys Ser Gly Lys Ser Ser Lys Ser Ser Lys | |
| | Ser Ser Gly Arg Arg Pro Leu Phe Gly Val Ser Gln Leu Ser Glu Gly | |
| | Ile Ala Ala Gly Tyr Ala Lys Ser Ser Gly Arg Ser Ser Gln Gln Ala | |
| | Val Gly Ser Trp Met Pro Val Ala Ala Ala Cys Ile Leu Gly Ala Leu | |
| | Ser Phe Phe Leu Asn | |
| 4 | Val Lys Val Lys Val Lys Val Lys Val Pro Pro Thr Lys Val Glu Val | Synthetic silaffin protein |
| | Lys Val Lys Val | |
| 5 | Val Lys Val Ser Val Lys Val Ser Val Pro Pro Thr Lys Val Ser Val | Synthetic silaffin protein |
| | Lys Val Ser Val | |

### BRIEF DESCRIPTION OF THE FIGURES

The above-mentioned aspects of the present disclosure and the manner of obtaining them will become more apparent, and aspects thereof will be better understood by reference to the following description of the embodiments of the disclosure, taken in conjunction with the accompanying drawings, figures, schemes, formula, and the like, wherein:

FIG. 1a is a scanning electron micrograph of a diatom illustrating silification of the cell wall.

FIG. 1b is a greater magnified image of a region of FIG. 1a illustrating patterning associated with the silicification of the diatom cell wall.

FIG. 2 is a flow chart describing one embodiment for practicing the present disclosure.

FIG. 3a is a mammalian cell unexposed to a non-naturally occurring bioinorganic material-rich environment.

FIG. 3b is a scanning electron micrograph of a mammalian cell in suspension after association of a non-naturally occurring bioinorganic membrane to the cell surface.

FIG. 4 depicts an embodiment of the disclosure in which a peptide is directly associated with the cell surface.

FIG. 5 depicts an embodiment of the disclosure in which a peptide is indirectly associated with the cell surface.

FIG. 6 is an illustration of a bioreactor having a cellular biofilm cultured thereon with a non-naturally occurring bioinorganic membrane associated with a surface of the cellular biofilm.

FIG. 7 is an image of living cells, stained with CellTracker™ green live stain following after association of a non-naturally occurring bioinorganic membrane to the cell surface.

FIG. 8 is a graph of proton flux measurements of living cells following after association of a non-naturally occurring bioinorganic membrane to the cell surface.

FIG. 9a is a scanning electron micrograph of Pseudomonas aeruginosa cells prior to exposure to a non-naturally occurring bioinorganic material-rich environment.

FIG. 9b is a scanning electron micrograph of Pseudomonas aeruginosa cells after after association of a non-naturally occurring bioinorganic membrane to the cell surface.

FIG. 10a is a scanning electron micrograph of Nitrosomonas europaea cells prior to exposure to a non-naturally occurring bioinorganic material-rich environment.

FIG. 10b is a scanning electron micrograph of Nitrosomonas europaea cells after after association of a non-naturally occurring bioinorganic membrane to the cell surface.

FIG. 11a is a graph presenting oxygen flux measurements of Pseudomonas aeruginosa cells during biomineralization.

FIG. 11b is a graph presenting oxygen flux measurements of Nitrosomonas europaea cells during biomineralization.

FIG. 12 is a graph presenting glucose influx patterns of non-naturally occurring silica entrapped INS-1 cells, non entrapped INS-1 cells and HIT β cells.

FIG. 13a is a transmission electron micrograph illustrating biomineralization of Max8 peptide associated INS-1 cells.

FIG. 13b is a magnified and localized transmission electron micrograph of a region of the cellular membrane of the INS-1 cell of FIG. 13a.

FIG. 14a is scanning electron micrograph illustrating biomineralization of a silaffin associated INS-1 cell.

FIG. 14b is lower magnification scanning electron micrograph of the INS-1 cells of FIG. 14a.

### DETAILED DESCRIPTION OF THE DISCLOSURE

The embodiments of the disclosure presented and/or described below are not intended to be exhaustive or to limit the precise forms disclosed in the following detailed description. Rather, the embodiments are chosen and described so that others skilled in the art may appreciate and understand the principles and practices of various aspects and embodiments discussed herein.

Unless specifically stated otherwise, as used herein, the term “about” refers to a range of plus or minus (+/−) 10% (e.g., 1.0 encompasses the range of values from 0.9 to 1.1).

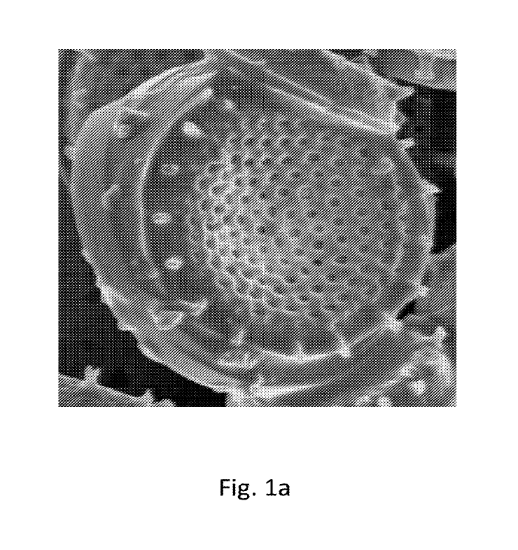

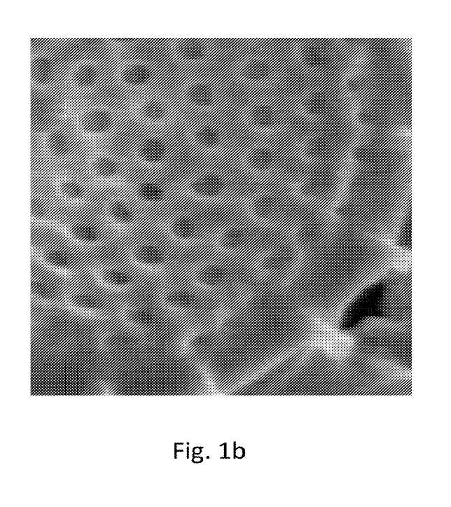

With reference to FIG. 1a, a scanning electron micrograph (SEM) illustrates the silification of the cell wall of a diatom. For many years, it has been known that the cell walls of diatoms are comprised of amorphous silica. Further, and with reference to FIG. 1b, the silification of diatom cell walls is known to comprise unique and functional nano and micro porous patterning. Surprisingly, however, the materials and methods of the instant disclosure provide for the formation of similar bioinorganic membranes onto the cell surfaces of evolutionary distinct organisms such as mammalian eukaryotic cells, for example.

The astonishing patterns found on the silica rich cell walls of many diatoms are clues to the utility of these structures. These patterns are channels through the silica rich protective naturally occurring cell walls that enable these organisms to freely exchange nutrients and waste material with their environments. The naturally occurring cell wall of the diatoms provide functionalities far superior to cells that are merely encased, entrapped or coated with materials such as silica rich layers. The patterns are the result of the deposition of silica facilitated by the association of specific moieties on the diatoms cell membrane that are evolved to interact with silica and to direct the formation of silica surface.

Many of these moieties in diatoms are polypeptides that include stretches that are lysine rich and that interact with dissolved silicic acid. These polypeptides include a class of proteins referred to as silaffins. For a further discussion of the purification and characterization of such proteins, please see Poulsen and Kroger, JBC, Vol. 279. No. 41, October 8, pp. 42993-42999. Amino acid sequences for 3 of the proteins disclosed in Poulsen and Kroger can be found listed herein as SEQ ID NO. 1, SEQ ID NO. 2 and SEQ ID NO. 3. Some of the embodiments of the instant invention include associating silaffins with the surfaces of either prokaryotic cells or eukaryotic cells, other than diatoms, and under suitable conditions, produce viable cells that include a patterned, non-naturally occurring bio-membrane having a structure that is directed by the association of the silaffins with various moieties such as proteins, carbohydrates or lipids that are present on the cellular membranes of the cells.

Still other embodiments of the invention include associating synthetic polypeptides, such as the MAX8 peptide (SEQ ID NO. 4) disclosed in Altunbas, et al, AcsNANO, Vol. 4, No. 1, pp. 181-188 (2010), with the surface of a cell (that does not naturally form a biomineral rich cell membrane) in order to facilitate the formation of a biomineral rich membrane having a pattern directed by moieties on the cell surface that interact with the peptide. These cells further remain viable.

Still other embodiments include polypeptides such as the one disclosed herein as SEQ ID NO. 5, which has physio-chemical properties similar to MAX8. The peptide of SEQ ID NO. 5 is designed to be less cytotoxic than MAX8 but still able to augment the cell surface directed formation of a biomineral rich membrane around, at least, a portion of cell surface that does not form biomineral rich cell walls in nature.

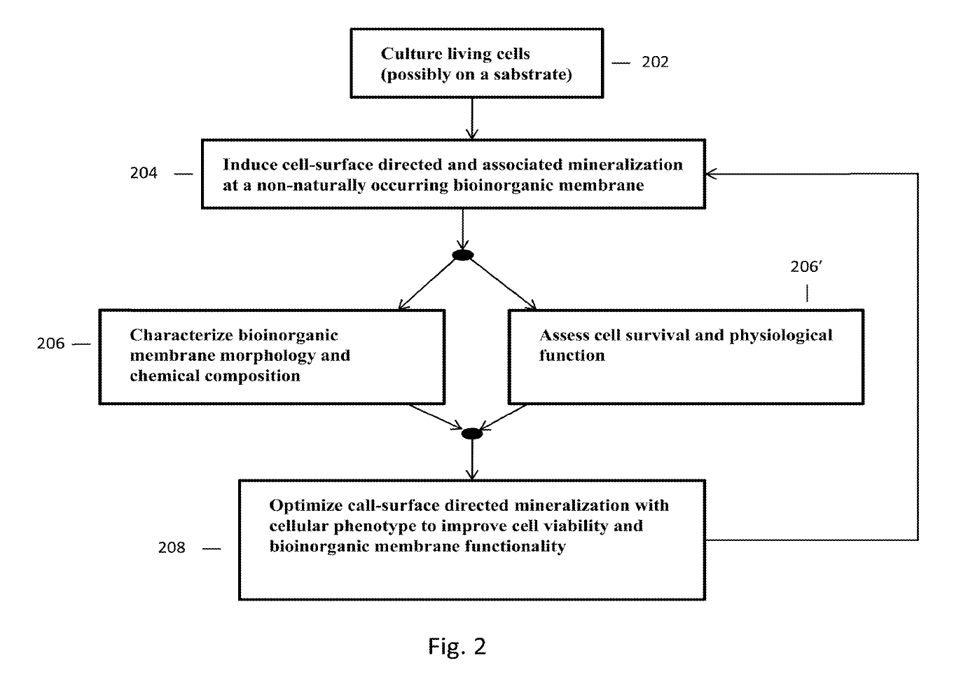

Broadly, the present disclosure provides materials and methods for cell-surface directed association of non-naturally occurring bioinorganic membranes with the cell surface of living cells which do not form biomineral rich cell walls in nature. Referring to FIG. 2, flow chart 200 is illustrated, providing a general description of one embodiment of the disclosure of a process for the formation of a biomineral rich membrane on the surface of almost any cell. As illustrated at step 202, living cells are cultured. As described herein, living cells include cells of organisms evolutionarily distinct from diatoms, including prokaryotes, such as Pseudomonas aeruginosa and Nitrosomonas europaea, and eukaryotic cells, such as mammalian pancreatic β-islets cells. Further, according to an embodiment of the present disclosure, the living cells may be cultured on the surface of a structure (as opposed to suspended cells in media).

With reference to step 204, association of the non-naturally occurring bioinorganic membrane with the cell-surface is induced. As will be explained in further detail below, induction of this association may occur in various manners, but in general accordance with the disclosure, involves introduction of the living cells to a bioinorganic material-rich (or even saturated) environment (such as a silica-rich buffer).

Further, as used herein, non-naturally occurring biomineral membranes are mineral rich structures, generally having a pattern that includes pores and are associated with cells that are not associated with such biomineral rich structures in nature. In some embodiments, the biomineral membrane may exist in nature as, for example, a silica rich cell wall in a diatom, but, as used herein, the same biomineral composition is defined as non-naturally occurring because in its inventive embodiment it is associated with a cell type, such as a prokaryotic cell, or an animal cell, or a higher plant cell, that it is not associated with in nature.

Still another wholly unexpected embodiment is that biomineral rich cell membranes (pseudo cell walls) can be formed on surfaces of cells such as Pseudomonas, stem cell like P19 murine embryonic carcinomas and mouse pancreatic β-islets cells by maintaining these cells in contact with a biomineral rich buffer for a length of time, even in the absence of the addition of exogenous polypeptides, such as silaffins. As illustrated in more detail herein, these cells remain viable and are able to exchange nutrients and products produced by the living cells with their environments. Without wishing to be bound by any theory, it appears as if naturally occurring moieties on the surface of cells, such as pseudomonas P19s, and β-islets, can direct the formation of a non-naturally occurring biomineral rich membrane (pseudo cell wall) by their ability to accumulate a biomineral such as silica from a biomineral rich buffer.

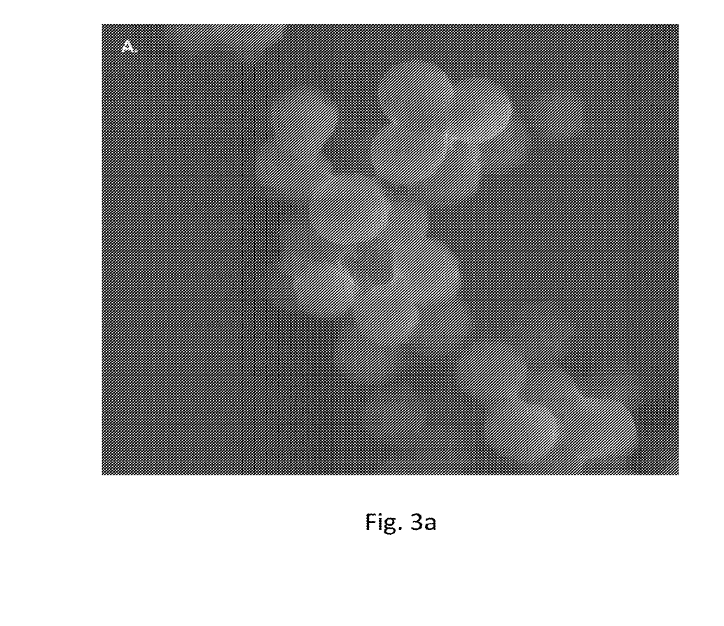

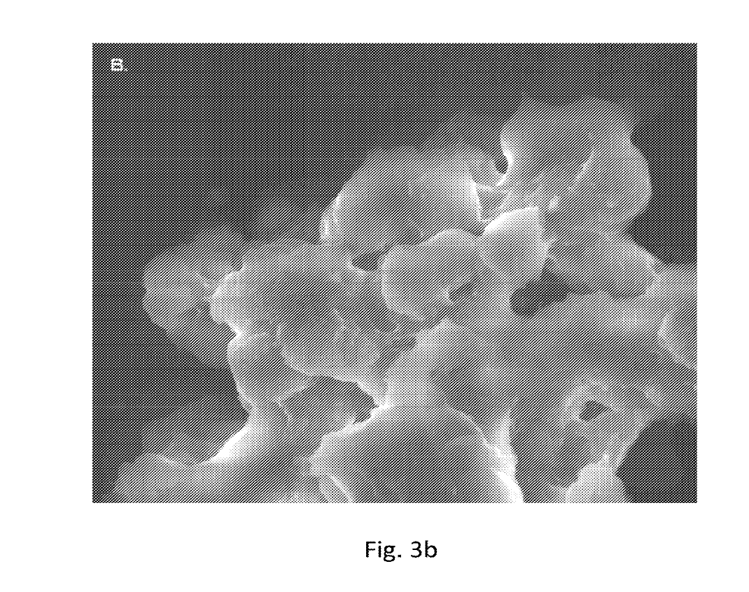

Referring next to steps 206 and 206′ of FIG. 2, following mineralization of the non-naturally occurring bioinorganic membrane to the cell surface, analysis of the living cells may be performed. With reference to step 206 specifically, characterizations of the bioinorganic membrane may be performed, including characterization of the membrane morphology and chemical composition. For example, scanning electron microscopy, and the like, may be performed as in FIGS. 3b, 9b, 10b, 13a, 13b, 14a, and 14b, in order to analyze porosity and micro- (and nano)-patterning of the associated bioinorganic membrane.

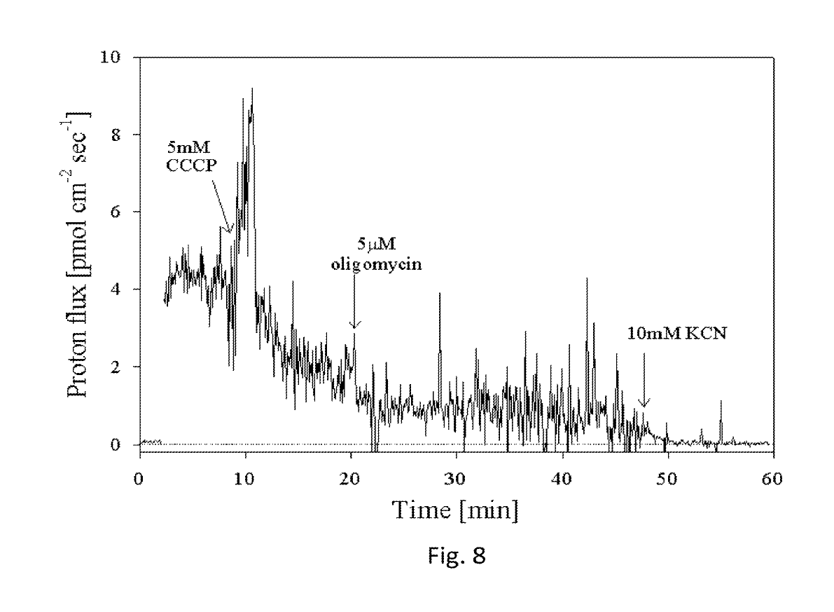

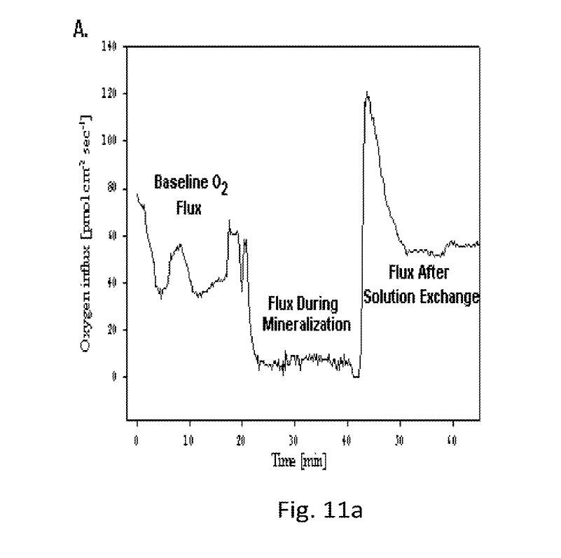

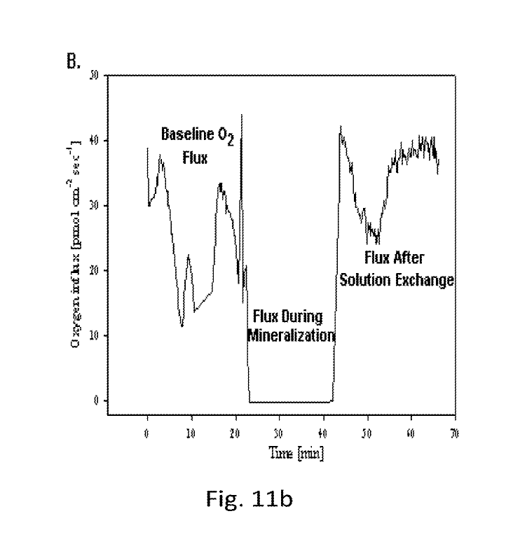

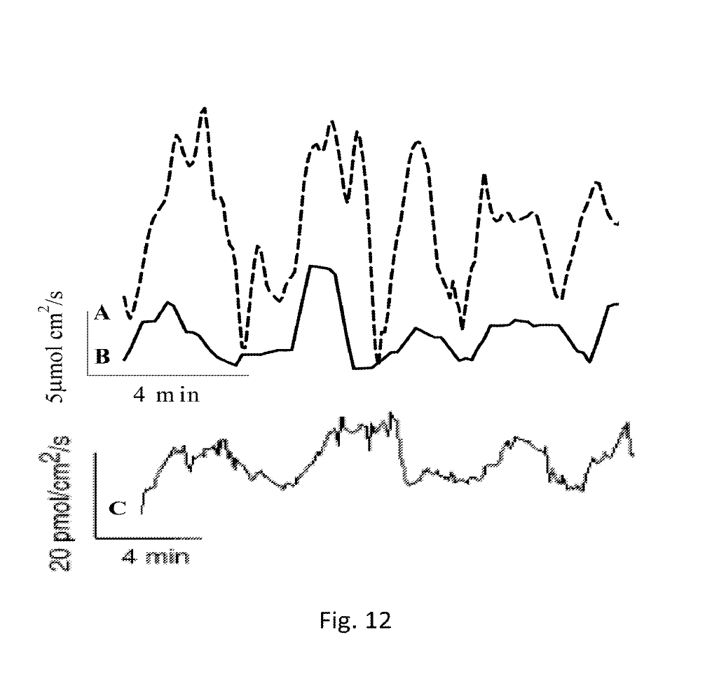

With reference to step 206′ specifically, cell survival and physiological functionalities of the living cells having the associated bioinorganic membrane may also be assessed. For example, proton (FIG. 8), oxygen (FIGS. 11a and 11b), and glucose (FIG. 12) flux measurements may be recorded and analyzed for the living cells following association of the bioinorganic membrane with the cell surface.

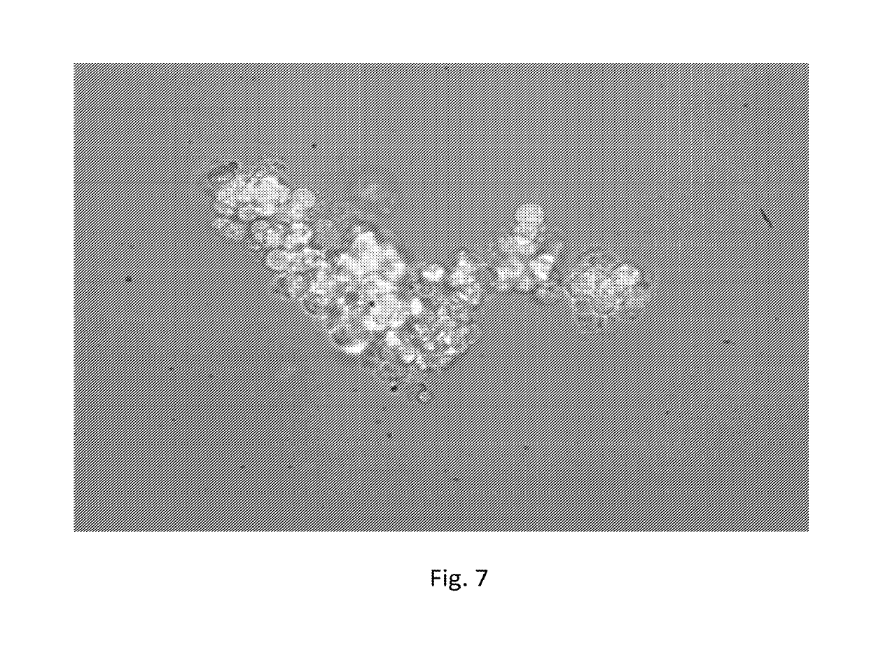

Next, and with reference to step 208 of FIG. 2, optimization of the materials and methods disclosed herein may be performed. For example, the biomineralization method of the present disclosure may be adjusted in regard to the living cells' phenotype and viability (FIG. 7), as well as the associated membrane functionality. Optimization of the disclosed materials and methods include, but is not limited to, varying the pH of the biomineralization buffer, varying the biomineral concentration within the biomineralization buffer, altering the exposure time of the living cell to the biomineralization buffer, varying the living cell density and life-cycle time point in regard to time of exposure, and altering the reaction temperature, for example.

According to one embodiment of the present disclosure, a non-naturally occurring bioinorganic membrane may be associated with a cell surface of a living cell (evolutionary distinct from diatoms) by exposing the cell surface to a biomineralization buffer (rich or saturated in the biomineral). In some embodiments the silica solution is acidic before it is introduced into the physiological buffer. The resultant neutralization of the silica increases the rate of polycondensation of the silicate into an amorphous state that is well-suited for biomineral deposition. For example, FIG. 3b provides a scanning electron micrograph of a mammalian cell having silica associated with the cell surface following exposure to a silica-rich buffer. To provide a comparison, FIG. 3a illustrates a mammalian cell (at the same magnification) which was not exposed to any non-naturally occurring bioinorganic material-rich environment. As is easily observed in regard to FIG. 3b, a bioinorganic membrane (comprised specifically of silica) has mineralized in association with the cell surface. It should also be noted that cellular activity of both the exposed (FIG. 3b) and unexposed (FIG. 3a) mammalian cells was confirmed through intercellular esterase staining (FIG. 7) and proton flux measurements (FIG. 8).

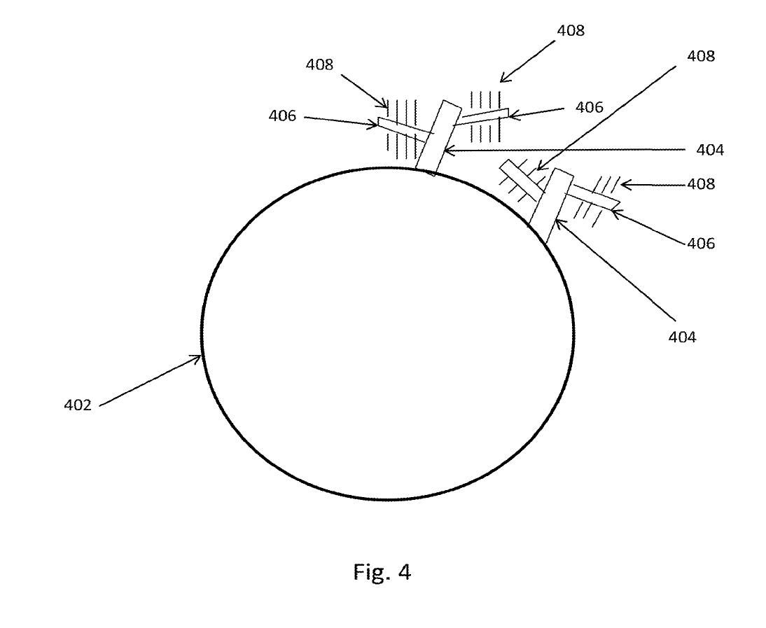

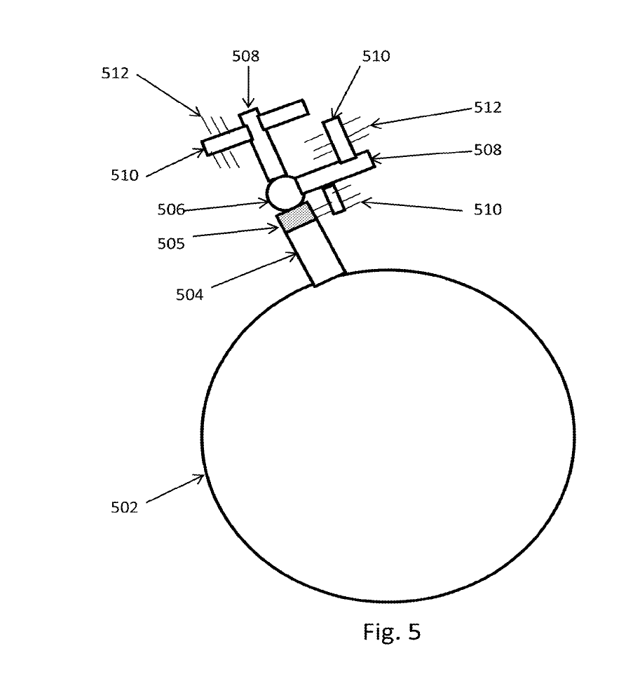

Another embodiment of the present disclosure, represented in the schematics of FIGS. 4 and 5, involves modification of living cells 400, 500 through attachment to cell surfaces 402, 502 of (one or more) peptides 404, 508 having at least one polyamine group 406, 510 attached thereto. Described herein, the exposure of living cells 400, 500 (with attached peptides 404, 508) to a buffer rich (or saturated) with a non-naturally occurring bioinorganic material (having a net negative charge) produces association of a bioinorganic membrane with the cell surfaces 402, 502 of living cells 400, 500.

With reference to FIG. 4, direct attachment (used herein as including binding, linking and associating and reacting with) peptide 404 is depicted. Also shown in FIG. 4, peptide 404 has at least one polyamine group 406 attached thereto. According to the embodiment of the present disclosure depicted in FIG. 4, when a non-naturally occurring bioinorganic material-rich environment (in a buffer) is introduced to living cell 400, non-naturally occurring bioinorganic material 408 associates with polyamine group 406 to form a membrane (at least partially) associated with cell surface 402.

Similar to FIG. 4, FIG. 5 also provides a schematic illustrating an embodiment of the present disclosure involving the attachment to the cell surface of a peptide with a polyamine group attached thereto. Unlike FIG. 4, however, FIG. 5 depicts an embodiment of the disclosure utilizing indirect attachment of at least one peptide 508 (having at least one polyamine group 510 attached thereto) to cell surface 502 of living cell 500. According to the depicted embodiment, indirect attachment of peptide 508 comprises binding of ligand 504 (including reactive group 505) to cell surface 502. Intermediate group 506 binds to reactive group 505 of ligand 504, wherein peptide 508 binds to intermediate group 506. As depicted in the embodiment of the present disclosure of FIG. 4, peptide 508 has at least one polyamine group 510 attached thereto, which, when introduced to a non-naturally occurring bioinorganic material-rich environment associates with the non-naturally occurring bioinorganic material 408 forming a membrane (at least partially) associated with cell surface 502. Reagents that can be used to attach various groups to the cell surface moieties include, but are not limited to, various antibodies. Judicious selection of such binding reagents can be used to control the structure of the biomineral mineral membrane so formed.

In accord with the instant disclosure, peptides 404, 508 (including polyamine groups 406, 510 attached thereto) have an overall net positive charge under the buffer conditions utilized herein. In general, peptides 404, 508 may comprise any one (or combination thereof) of a silaffin protein, silicatein protein, a polyamine rich naturally occurring cell surface peptide, a synthetic polyamine rich peptide, a silaffin derivative, a silicatein derivative, thiolayted peptides, peptides that have free hydroxyl groups including amino acids such as serine, threonine, hydroxyproline, and SEQ ID NO. 1, SEQ ID NO. 2, SEQ ID NO. 3, SEQ ID NO. 4, SEQ ID NO. 5, and the like.

Silaffin peptides within the scope of the present disclosure include, but are not limited to, silaffin proteins derived from Thalassiosira pseudonana, Coscinodiscus wailesii, Coscinodiscus concinnus or any combination thereof. Additionally, silaffin peptides may be isolated from any diatom, produced recombinantly, or produced synthetically.

It should be understood that embodiments of the present disclosure depicted in FIGS. 4 and 5, involving association of peptides 404, 508 to cell surfaces 402, 502 of living cells 400, 500, provide for enhanced control in design of bioinorganic membrane. For example, bioinorganic membrane density, porosity and micro (and nano) patterning may be adjusted through practice of the disclosed embodiments of the instant materials and methods depicted in FIGS. 4 and 5. In contrast to the embodiment described by FIGS. 4 and 5, the embodiment depicted in FIG. 3b (in which the bioinorganic membrane associates to endogenous proteins) illustrates less patterning of the protein architecture.

Further, in some embodiments, association of the silaffins to the cell surface can be accomplished by taking advantage of integrin/ligand binding interactions. Peptides with affinities for specific cell surface integrins can be readily produced synthetically or in transgenic bacteria. Simple chemical modification can be employed to attach a thiol (—SH) group to the terminus of the peptide chain. When introduced into solution, the peptides will bind to surface integrin receptors, studding the cell with gold binding thiol groups. Gold nanoparticles can then be added to the media and allowed to attach to the thiol groups studding the cell. Silaffins, produced by transgenic diatoms and chemically modified to express a thiol group on the peptide chain terminus, can then be introduced. The gold affinity of the thiol modified silaffins will induce aggregation onto the nanoparticles. Once the cell has been decorated with silaffins, immersion into silica rich solution will result in the silaffin governed nanopatterning of a silica shell. Alternative binding strategies can be applied to the same experimental motif Biotinylated peptide termini could be chemically produced to couple with avidin coated microbeads. The more direct approach of creating a ligand/silaffin fusion protein in the recombinant diatom could also be used to associate the silaffin to the cell membrane; however, the relative ease of cellular adaptability would be compromised. Nanoparticle junctions prevent the need to create new recombinant silaffin proteins for every ligand explored. Self assembly of silaffins onto a nanoparticle would also allow for peptide concentration dependant morphological structure control. Direct ligand/silaffin binding would place an upper limit on silaffin concentration to the number of integrins expressed on the membrane. Precise control of silaffin concentration will be necessary in order to manipulate the pore morphology and diffusional characteristics of the coating.

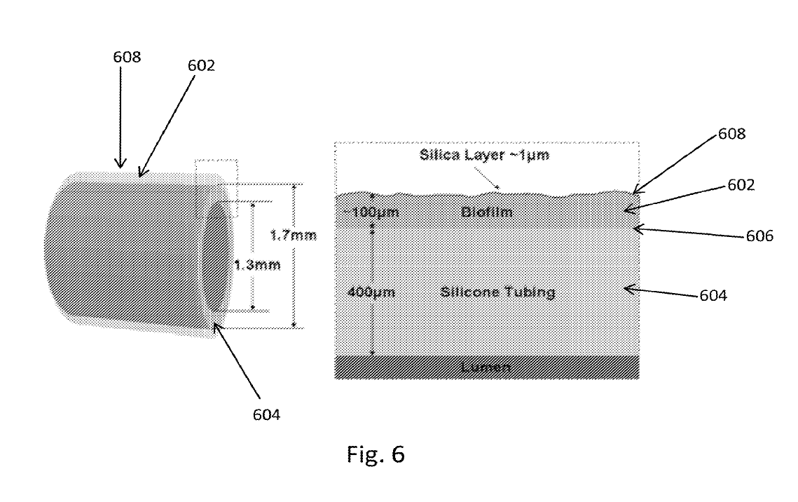

Referring next to FIG. 6, another embodiment of the present disclosure is shown. The embodiment depicted in FIG. 6 comprises the association of bioinorganic membrane 608 to biofilm 602 which is associated with surface 606 of structure 604 (depicted herein as a hollow silicone tube as may be used in catheters or the like). As used herein, a “structure” can be any material, including but not limited to a fiber stainless steel, plastic (or a can alloy or composite thereof) and tubing.

Thus the present disclosure also provides materials and methods for the association of a non-naturally occurring bioinorganic membrane 608 to the cell surface of living cells which are associated with a structure. Such embodiments employ the additional steps relating to associating (or culturing) cells onto a structure. Embodiments of the present disclosure as depicted in FIG. 6 are useful in fields such as medical devices, drug discovery and targeting applications, and transplant therapies, for example.

Unlike the artificial deposition strategies (which create bioinorganic membranes around cells) of current technologies, embodiments of presently disclosed non-naturally occurring bioinorganic membranes, formed by the cell surface directed and associated materials and methods disclosed herein, are biocompatible, strong, and chemically resistant. Further, the bioinorganic membranes generated by the present disclosure possess relatively rapid (compared to current technologies) rates of molecular diffusion critical for maintenance of cell viability.

Embodiments of the materials and methods described above allow for uses in the association of non-naturally occurring bioinorganic membranes with the surface of living cells, both prokaryotic and eukaryotic. Additionally, embodiments of the present disclosure allows for uses in sensors and adaptive drug delivery devices as well as for the implantation of foreign cellular material into a host without the need for global suppression of the immune system of the host. Further, the bioinorganic membrane disclosed herein can be used for regulation of the release of a wide range of molecules in products such as pharmaceutical agents, nutrients, gasses, and biological products. Even further, methods of the present disclosure may also be employed in applications with structures other than living cells. For example, the present disclosure may be used with drug carrying structures, such as hydrogels, polymer particles, liposomes, and micelles in order to create controlled release drug delivery devices.

### EXAMPLES

### Example 1

### Cell Mediated Formation of Silica-Based Biomineral Membrane on Endogenous Cell Surface Proteins

Materials and Methods: A biomineralization solution was prepared by hydrolysis of tetramethyl orthosilicate (TMOS) in a weakly acidic aqueous solution. The methanol byproduct of the hydrolysis reaction was removed by rotary evaporation. Suspended mouse (P19) cells were then exposed to media containing the mildly acidic silica-rich solution, resulting in the polycondensation of a biomineral membrane. The solution was then diluted prior to bulk gelation. Tetramethyl orthosilicate (TMOS, Sigma-Aldrich) was hydrolyzed in a 1:16 mol ratio (TMOS:H₂O) deionized water solution using 1 μl of 0.04 molar hydrochloric acid initiator per 1 g of solution. The mixture was stirred vigorously for 10 minutes until clear. The methanol produced by the hydrolysis reaction was removed from the solution by rotary evaporation under vacuum at 45° C. (30% reduction in solution volume). The resulting saturated silica solution was refrigerated prior to use or used immediately. Biomineral layer formation was induced by exposing cells to a α-MEM media solution supplemented with 30 μl per ml of the previously prepared saturated silica solution and 50 μl per ml phosphate buffered saline. The cells were incubated in this solution for 10-30 minutes (longer times producing thicker mineral deposits). After mineralization, the solution was removed and fresh silica free media was reintroduced to the cells.

Results: With specific reference to FIGS. 3a and 3b, the suspended mouse (P19) cells which were exposed to the biomineralization buffer (FIG. 3b) and control (unexposed) cells (FIG. 3a) were analyzed with a scanning electron microscope. As can be seen by FIGS. 3a and 3b, the exposed cells exhibited silica polycondensation on the cell surface whereas the unexposed cells, as expected, did not exhibit any polycondensation. The cells are tested for metabolic active using MitoTracker Mitochondrial stain and had in tact cellular membranes using CellTracker live cell stain. We also detected oxygen flux from these encapsulated cells.

Further, and with reference to FIGS. 7 and 8, cellular activity of the exposed cells was assayed. As depicted in FIG. 7, the exposed cells were stained with CellTracker™ green live stain, demonstrating the exposed cells retained intercellular esterase activity. The graph of FIG. 8 further confirmed the exposed cells retained cellular activity by demonstrating the proton flux (measured at the biomineral membrane) increased following addition of 5 μM-CCCP (a proton ionophore). CellTracker staining procedure provided by the manufacturer (Invitrogen) is used in order to quantify biophysical flux of substrate (glucose or NH₄⁺), O₂, and H⁺ were measured using the self-referencing (SR) technique from (Porterfield 2007; McLamore, Porterfield et al. 2009). SR converts concentration sensors into dynamic biophysical flux sensors for quantifying real time transport in the cellular to whole tissue domain, and has been used in many fields, including: agricultural (Porterfield, Kuang et al. 1999; Gilliham, Sullivan et al. 2006), biomedical (Land, Porterfield et al. 1999; Zuberi, Liu-Snyder et al. 2008), and environmental (Sanchez, Ochoa-Acuna et al. 2008; McLamore, Porterfield et al. 2009; McLamore, Zhang et al. 2010) applications. SR discretely corrects for signals produced by ambient drift and noise by continuously recording differential concentration (ΔC) while oscillating a microsensor between two locations separated by a fixed excursion distance (ΔX), and calculating analyte flux using Fick's first law of diffusion (Kuhtreiber and Jaffe 1990). SR sensors were used to non-invasively quantify oxygen and substrate flux using established methods (McLamore, Porterfield et al. 2009). Briefly, oxygen flux was measured using a SR optical oxygen sensor, which was constructed by immobilizing an oxygen-quenched fluorescent dye (platinum tetrakis pentafluoropheynl porphyrin) on the tip of a tapered optical fiber. Substrate (glucose) flux was amperometrically measured using a glucose biosensor that was fabricated by entrapping glucose oxidase within a Nafion/carbon nanotube layer on the tip of a platinized Pt/Ir wire (McLamore, Shi et al. 2010).

Experiment 1, described above, demonstrates both that cells which are evolutionary distinct from diatoms (do not form biomineral membranes by extracting anionic biominerals from the environment) surprisingly form such membranes after exposure to the biomineralization buffer disclosed herein. Further, Experiment 1 demonstrates these cells surprisingly retain their cellular activity and functionality, thus demonstrating the associated membrane disclosed herein possess mesoporosity enabling necessary cellular transport and diffusion of cellular material.

### Example 2

### Formation of Silica-Based Biomineral Membrane on Biofilms

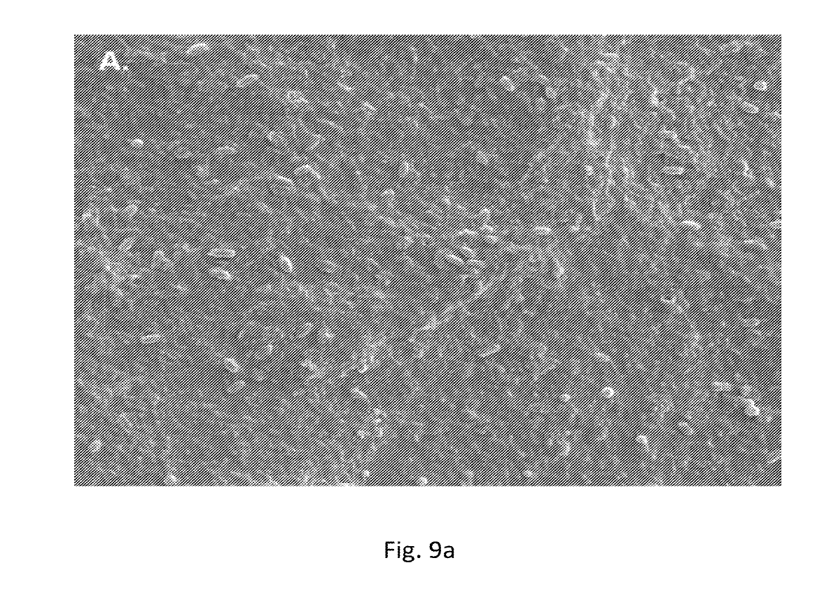

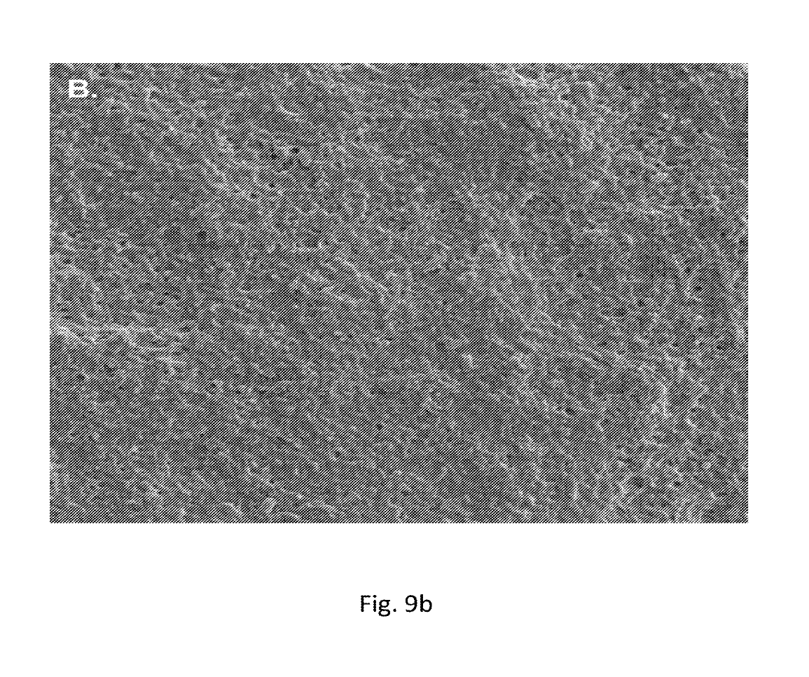

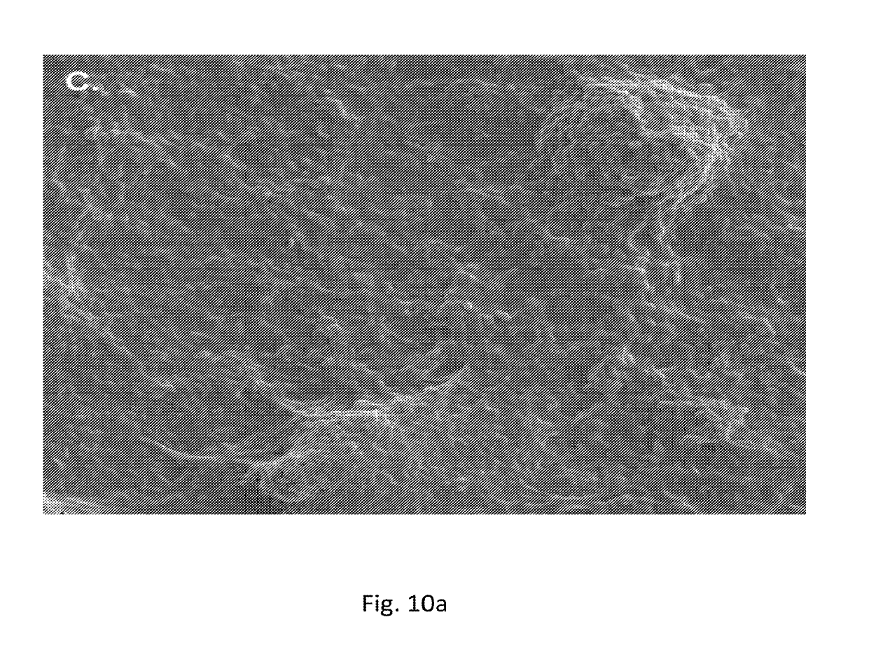

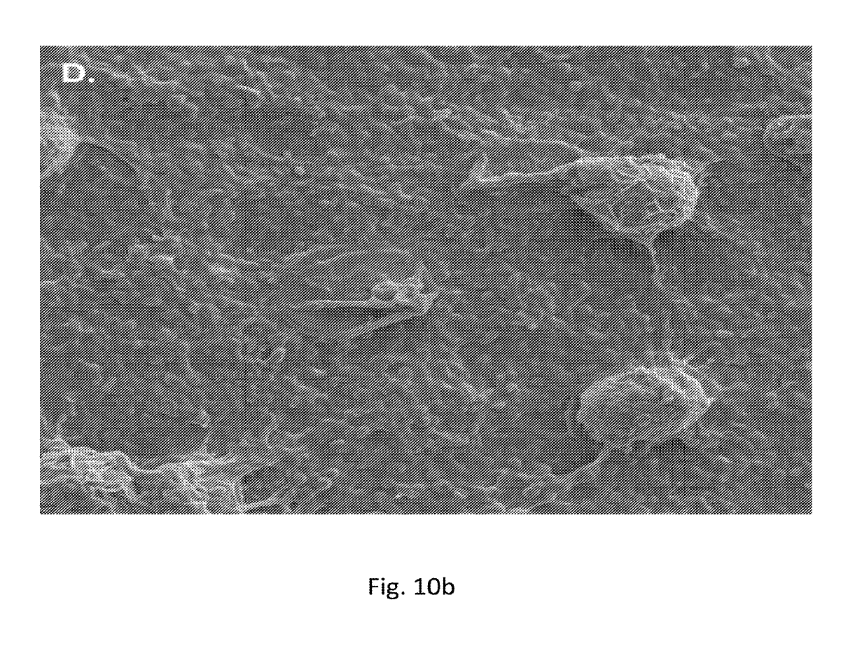

Materials and Methods: Mucopolysaccharide-rich P. aeruginosa and N. europaea biofilms were immersed in a mildly acidic silica-rich biomineralizing buffer. P. aeruginosa PA01 (ATCC 97) was obtained from American Type Culture Collection (Manassas, Va.), and biofilms were grown at 37° C. in modified glucose media (10 mM glucose, 50 mM HEPES, 3 mM NH₄Cl, 43 mM NaCl, 3.7 mM KH₂PO₄, 1 mM MgSO₄, and 3.5 μM FeSO₄). N. europaea (ATCC 19718) was obtained from ATCC, and biofilms were grown in ATCC medium 2265 (25.0 mM-(NH₄)₂SO₄, 43.0 mM-KH₂PO₄, 1.5 mM-MgSO₄, 0.25 mM-CaCl₂, 10 μM-FeSO₄, 0.83 μM-CuSO₄, 3.9 mM-NaH₂PO₄, and 3.74 mM-Na₂CO₃). The biofilms were mineralized in freshly filtered growth medium supplemented with 25 μl per ml of the saturated silica solution described previously for ˜20 min prior to media exchange. Scanning electron microscopy images of the biofilims prior to and after membrane formation are presented in FIGS. 9a, 9b, 10a, and 10b. Surprisingly, it was observed that P. aeruginosa biofilms form relatively flat, smooth structures, while N. euoropaea form morphologically heterogenous surfaces with fruiting bodies (Purevdorj-Gage, Costerton et al. 2005).

The SEMS were taken after fixing the biofilms on the membrane (FIG. 6) using a 4% glutaraldehyde/sterile phosphate buffer solution for 1 hour. The samples were then soaked in deionized water for 15 minutes, followed by serial dehydration in ethanol solutions (25%, 50%, 75%, 90%, and 100% respectively). Upon removal from the final ethanol wash, the samples were placed in a partially enclosed polystyrene dish and allowed to dry slowly under ambient conditions for 8 hours. Samples were then placed in a desiccating chamber prior to SEM imaging. As far as the encapsulation, we dipped the biofilms in the mineralizing solution for 20 minutes and then put it back in fresh media

Results: As is observed in FIGS. 9a, 9b, 10a, and 10b, both biofilms, following formation of a silica membrane layer, retained their respective morphology. Specifically, FIGS. 9a and 9b illustrate Pseudomonas aeruginosa prior to (9a) and after (9b) exposure to mildly acidic silica-rich biomineralizing buffer. FIGS. 10a and 10b illustrate Nitrosomonas europaea prior to (10a) and after (10b) exposure to silica precursor rich solutions.

With reference to FIGS. 11a and 11b, oxygen flux measurements were conducted during the biomineralization process to determine the physiological impact of biofilm exposure to mineralizing solutions. Oxygen uptake was monitored for 10 minutes to determine baseline aerobic respiratory level. The media was then carefully removed and filtered media containing 25 μl per ml enriched silica solution was added. The samples were allowed to rest in the saturated silica for 20 minutes in order to encapsulate the biofilm. Oxygen flux measurements were monitored throughout the biosilicification process. After 20 minutes, the solution was again carefully removed and replaced with fresh silica free medium to halt the biosilicification process. Oxygen flux measurements were then continuously recorded along the biofilm surface for 14 hours to monitor biofilm viability. Additional encapsulated biofilms were returned to the bioreactor and allowed to incubate for 30 and 90 days before flux analysis. As a control experiment, flux was measured in growth media, the solution was replaced with fresh growth media containing no silica, and physiological flux/viability measured. For all later experiments, substrate and/or O₂ flux were continuously measured at five positions along the surface of each biofilm for ten minutes unless otherwise indicated (2 mm in the lateral direction between each position). For data concerning physiological flux, all averages represent the arithmetic mean of at least ten minutes of continuous recording at five positions (n=3 replicates), and error bars represent the standard error of the arithmetic mean. As is shown in both graphs, biofilm oxygen flux reduced dramatically during biomineralization, but returned rapidly to baseline levels after solution exchange. Thus, while the cells appear to have been stressed during biomineralization (typically 10-20 minutes), the rapid return to pre-stressed levels shown in the graphs indicates that the cells recovered following formation of the respective silica layers.

Additionally, viability florescent staining (with STYO9 green) was also performed on both the P. aeruginosa and N. europaea biofilms (not depicted) (staining of control cells with and propidium iodide was also performed). The results of the staining analysis found no statistically significant variation between control and biomineralized cell populations. These results indicated that the silica matrix was sufficiently porous to allow for the diffusion of dissolved gasses and nutrients. Biophysical transport of nutrients and electron acceptors regulates synthesis and maintenance of cells within the biofilm and is limited by the concentration boundary layer formed at the biofilm-fluid interface. No significant change in oxygen flux, substrate flux, or stoichiometric metabolic ratio was observed after encapsulation (p<0.02, α=0.05), suggesting that cells survived the encapsulation process intact. No observable differences were noted at 10× magnification in stained samples analyzed using confocal microscopy. There were no large regions of lysed cells within the matrix (2 μm slices), which one would expect if diffusion limitations or nutrient transport was significantly altered by silica encapsulation.

### Example 3

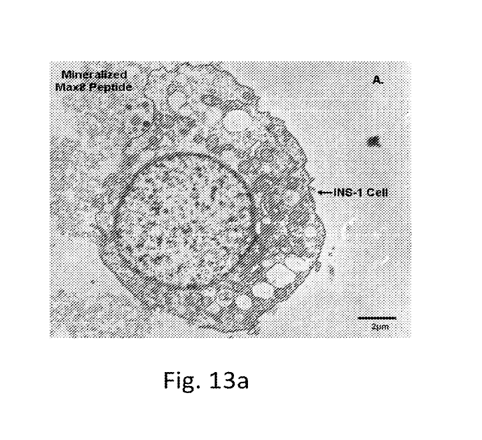

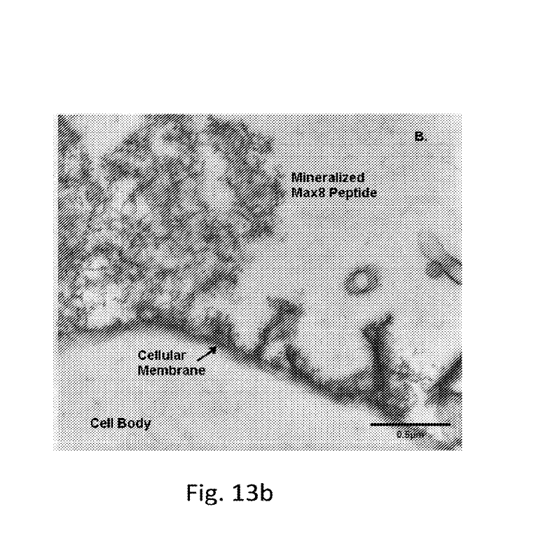

With reference to FIGS. 12, In a preliminary study of glucose flux from encapsulated cells, adherent rat pancreatic β (INS-1) cells were subjected to a biomineralizing solution. Glucose responsiveness was then assessed using a self-referencing glucose sensor according to Shi et al. and Porterfield (Porterfield 2007; Shi, Diggs et al. 2008) (FIG. 13). INS-1 cells demonstrate cyclic glucose intake prior to and after biomineralization which is similar to cyclic oxygen patterns in HIT β cells (Porterfield, Corkey et al. 2000). The cells were responsive to glucose stimulation, displaying regular influx patterns after bolus introduction of additional glucose and eventually stabilizing in a cyclic pattern with an average oscillation period (3.48±0.28 minutes) similar to that reported for HIT β cells (3.2 minutes) (Porterfield, Corkey et al. 2000).

Referring now to FIGS. 13a, 13b, INS-1 cells were exposed to the synthetic self assembling Max8 peptide. The peptide (20 mg per 10 ml media) was added to a cell suspension in serum free media (RPMI media supplemented 50 μl per ml phosphate buffered saline) and allowed to electrostatically adhere and assemble onto the exterior cellular membrane. An enriched silica solution was introduced in order to mineralize the fibrils (20 μl per ml of RPMI of the previously described saturated silica solution and 50 μl per ml phosphate buffered saline). The mineralized samples were then fixed for analysis using transmission electron microscopy (TEM). Cells were observed partially encased in a silicified fibrous mesh.

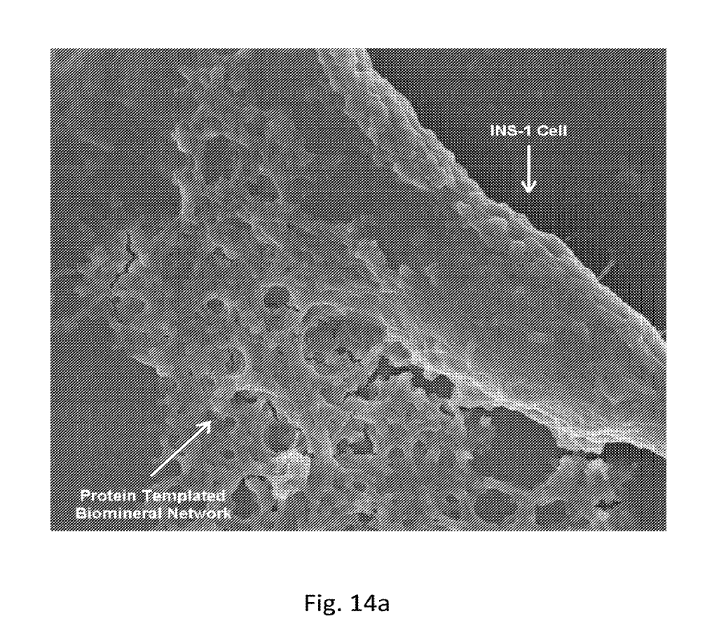

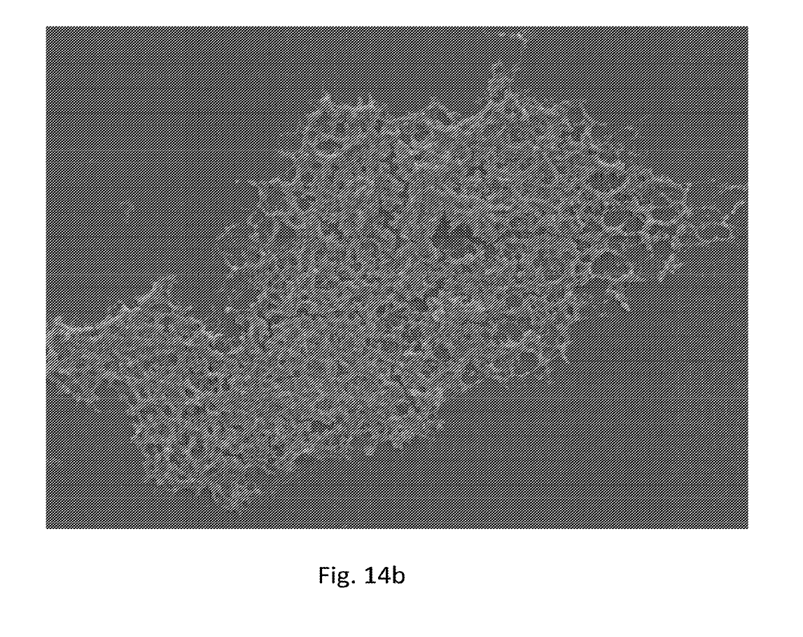

Referring now to FIGS. 14a, and 14b. Preliminary investigation of diatom protein templated silica biomineralization was conducted on the glucose responsive INS-1 β-cell line. Silaffin proteins from the diatom Thalassiosira pseudonana, were extracted by the method of Kroger et al. (Kroger, Deutzmann et al. 2000; Kroger, Lorenz et al. 2002). The proteins were then introduced to an adherent population of INS-1 cells and allowed to electrostatically adhere to the extracellular membrane. Following exposure to a mineralizing silica solution (RPMI media supplemented with 20 μl per ml of the previously described saturated silica solution and 50 μl per ml phosphate buffered saline), the cells were fixed and prepared for SEM analysis. Results of this study demonstrated biomineralization of the silaffin proteins. Micropatterened networks of silica (confirmed by EDS elemental analysis) were observed coating both cellular bodies and substrate.

### REFERENCES

Altunbas, A., N. Sharma, et al. (2010). “Peptide-Silica Hybrid Networks: Biomimetic Control of Network Mechanical Behavior.” Acs Nano 4(1): 181-188.
Gilliham, M., W. Sullivan, et al. (2006). “Simultaneous flux and current measurement from single plant protoplasts reveals a strong link between K+ fluxes and current, but no link between Ca2+ fluxes and current.” Plant Journal 46(1): 134-144.
Kroger, N., R. Deutzmann, et al. (2000). “Species-specific polyamines from diatoms control silica morphology.” Proceedings of the National Academy of Sciences of the United States of America 97(26): 14133-14138.
Kroger, N., S. Lorenz, et al. (2002). “Self-assembly of highly phosphorylated silaffins and their function in biosilica morphogenesis.” Science 298(5593): 584-586.
Kuhtreiber, W. M. and L. F. Jaffe (1990). “Detection of extracellular calcium gradients with a calcium-specific vibrating electrode.” J Cell Biol 110(5): 1565-1573.
Land, S. C., D. M. Porterfield, et al. (1999). “The self-referencing oxygen-selective microelectrode: Detection of transmembrane oxygen flux from single cells.” Journal of Experimental Biology 202(2): 211-218.
McLamore, E. S., D. M. Porterfield, et al. (2009). “Non-Invasive Self-Referencing Electrochemical Sensors for Quantifying Real-Time Biofilm Analyte Flux.” Biotechnology and Bioengineering 102(3): 791-799.
McLamore, E. S., J. Shi, et al. (2010). “A self referencing enzyme-based microbiosensor for real time measurement of physiological glucose transport.” Biosensors and Bioelectronics.
McLamore, E. S., W. Zhang, et al. (2010). “Membrane-Aerated Biofilm Proton and Oxygen Flux during Chemical Toxin Exposure.” Environmental Science & Technology 44(18): 7050-7057.
Porterfield, D. M. (2007). “Measuring metabolism and biophysical flux in the tissue, cellular and sub-cellular domains: Recent developments in self-referencing amperometry for physiological sensing.” Biosensors and Bioelectronics 22(7): 1186-1196.
Porterfield, D. M., R. F. Corkey, et al. (2000). “Oxygen consumption oscillates in single clonal pancreatic beta-cells (HIT).” Diabetes 49(9): 1511-1516.
Porterfield, D. M., A. X. Kuang, et al. (1999). “Oxygen-depleted zones inside reproductive structures of Brassicaceae: implications for oxygen control of seed development.” Canadian Journal of Botany-Revue Canadienne De Botanique 77(10): 1439-1446.
Purevdorj-Gage, B., W. J. Costerton, et al. (2005). “Phenotypic differentiation and seeding dispersal in non-mucoid and mucoid Pseudomonas aeruginosa biofilms.” Microbiology-Sgm 151: 1569-1576.
Sanchez, B. C., H. Ochoa-Acuna, et al. (2008). “Oxygen flux as an indicator of physiological stress in fathead minnow (Pimephales promelas) embryos: A real-time biomonitoring system of water quality.” Environmental Science & Technology 42(18): 7010-7017.
Shi, J., A. Diggs, et al. (2008). A bionanocomposite glucose microbiosensor for biomedical and plant physiology applications. Proceedings of the Institute of Biological Engineers Annual Conference, Chapel Hill, N.C.
Zuberi, M., P. Liu-Snyder, et al. (2008). Large naturally-produced electric currents and voltage traverse damaged mammalian spinal cord. 2: 17.

What is claimed is:

1. A method for forming a bioinorganic membrane, comprising the steps of:
attaching at least one polypeptide to a surface of at least one cell, wherein the cell does not form a bioinorganic membrane in nature, and wherein said at least one polypeptide associates with a non-naturally occurring bioinorganic material, wherein said non-naturally occurring bioinorganic material is rich in silica.

2. The method according to claim 1, wherein said polypeptide is one of:
directly attached to the surface of the cell by binding, linking, or associating said polypeptide with at least one group, wherein said at least one group is part of the cell; and
indirectly attached to the surface of the cell via a ligand, wherein the surface of the cell is attached to the ligand, wherein the ligand includes a reactive group, wherein the reactive group of the ligand binds to an intermediate group, and wherein the intermediate group includes a first portion and a second portion, wherein the first portion of the intermediate group is attached to the reactive group of the ligand and wherein the second portion of the intermediate group is attached to said polypeptide.

3. The method according to claim 1, wherein said polypeptide is selected from the group consisting of: a silicatein protein, a naturally occurring polyamine rich peptide, a non-naturally occurring polyamine rich peptide, a derivate of a silicatein, a derivative of a silaffin, a thiolated peptide, and a polypeptide that includes at least one free hydroxyl group.

4. The method according to claim 3, wherein the polypeptide that includes at least one free hydroxyl group includes at least one amino acid selected from the group consisting of: serine, threonine, and hydroxyproline.

5. The method according to claim 1, wherein said polypeptide is a silaffin.

6. The method according to claim 5, wherein the silaffin is derived from at least one species selected from the genera consisting of: Thalassiosira and Coscinodiscus.

7. The method according to claim 7, wherein the silaffin is derived from at least one species selected from the group consisting of: Thalassiosira pseudonana, Coscinodiscus wailesii, and Coscinodiscus concinnus.

8. The method according to claim 1, wherein said polypeptide is selected from the group consisting of: SEQ ID NO. 1, SEQ ID NO. 2, SEQ ID NO. 3, SEQ ID NO. 4, and SEQ ID NO. 5.

9. The method according to claim 1, wherein the at least one cell is selected from the group consisting of: a eukaryotic cell, a pancreatic beta cell, and a prokaryotic cell.

10. The method according to claim 9, wherein the prokaryotic cell is from one species of the genus Pseudomonas.

11. The method according to claim 1, further including the step of exposing the cells to a biomineralization solution, wherein the solution is mildly acidic and rich in silica.

12. The method according to claim 11, wherein the biomineralization solution is low in methanol and is formed by hydrolyzing tetramethyl orthosilicate in an acid.

13. A method for forming a bioinorganic membrane, comprising the steps of:
attaching a polypeptide to a surface of a bio-film, wherein the bio-film does not form a bioinorganic membrane in nature, and wherein said at least one polypeptide associates with a non-naturally occurring bioinorganic material, wherein said non-naturally occurring bioinorganic material is rich in silica.

14. The method according to claim 13, wherein said polypeptide is one of:
directly attached to the surface of the bio-film by binding, linking, or associating said polypeptide with at least one group, wherein said at least one group is part of the bio-film; and
indirectly attached to the surface of the bio-film via a ligand, wherein the surface of the bio-film is attached to the ligand, wherein the ligand includes a reactive group, wherein the reactive group of the ligand binds to an intermediate group, and wherein the intermediate group includes a first portion and a second portion, wherein the first portion of the intermediate group is attached to the reactive group of the ligand and wherein the second portion of the intermediate group is attached to said polypeptide.

15. The method according to claim 13, wherein said polypeptide is selected from the group consisting of: a silicatein protein, a naturally occurring polyamine rich peptide, a non-naturally occurring polyamine rich peptide, a derivate of a silicatein, a derivative of a silaffin, a thiolated peptide, and a polypeptide that includes at least one free hydroxyl group.

16. The method according to claim 13, wherein said polypeptide is a silaffin.

17. The method according to claim 13, wherein said polypeptide is selected from the group consisting of: SEQ ID NO. 1, SEQ ID NO. 2, SEQ ID NO. 3, SEQ ID NO. 4, and SEQ ID NO. 5.

18. The method according to claim 13, wherein the bio-film is a surface of a pancreatic islet.

19. A bio-structure, comprising:
at least one of a cell and a bio-film;
at least one polypeptide; and
a non-naturally occurring bioinorganic membrane;
wherein a first portion of said polypeptide is attached either directly or indirectly to a surface of the cell or the bio-film; and
wherein a second portion of said polypeptide is associated with a form of silica, wherein said polypeptide and the form of silica form part of the non-naturally occurring bioinorganic membrane.

20. The bio-structure according to claim 19, wherein said polypeptide is selected from the group consisting of: a silicatein protein, a naturally occurring polyamine rich peptide, a non-naturally occurring polyamine rich peptide, a derivate of a silicatein, a derivative of a silaffin, a thiolated peptide, and a polypeptide that includes at least one free hydroxyl group.

21. The method according to claim 20, wherein the polypeptide that includes at least one free hydroxyl group includes at least one amino acid selected from the group consisting of: serine, threonine, and hydroxyproline.

22. The method according to claim 19, wherein said polypeptide is a silaffin.

23. The method according to claim 22, wherein the silaffin is derived from at least one species selected from the genera consisting of: Thalassiosira and Coscinodiscus.

24. The method according to claim 23, wherein the silaffin is derived from at least one species selected from the group consisting of: Thalassiosira pseudonana, Coscinodiscus wailesii, and Coscinodiscus concinnus.

25. The method according to claim 19, wherein said polypeptide is selected from the group consisting of: SEQ ID NO. 1, SEQ ID NO. 2, SEQ ID NO. 3, SEQ ID NO. 4, and SEQ ID NO. 5.
